(19) United States
(12) Patent Application Publication
(10) Pub. No.: US 2026/0263748 A1
(43) Pub. Date: Sep. 10, 2026

(54) DEVICE FOR SECURING MEDICAL TUBING ON A PATIENT

(71) Applicants: Thomas R. GALBIERZ, Brentwood, TN (US); Michael A. GALBIERZ, St. Louis, MO (US)

(72) Inventors: Thomas R. GALBIERZ, Brentwood, TN (US); Michael A. GALBIERZ, St. Louis, MO (US)

(21) Appl. No.: 19/543,520

(22) Filed: Feb. 18, 2026

Related U.S. Application Data
(60) Provisional application No. 63/763,711, filed on Feb. 26, 2025.

(51) Int. Cl. A61M 25/02

(52) U.S. Cl. CPC A61M 25/02; A61M 2025/0226; A61M 2025/0253; A61M 2025/0266

(57) ABSTRACT

Described herein is a tube-securing device for securing medical tubing in place on a patient. The tube-securing device comprises a body adapted to be adhered to skin of a patient and has at least one tube securing element adapted to retain medical tubing in place relative to the tube-securing device. The device is formed from a material having an adhesive lower surface and a napped upper surface. The at least one tube-securing element comprises one of: (1) a finger having a fixed end and a free end, the finger being pivotal relative to the body about the fixed end; (2) a finger mounted to, and extending from, a fastener, the fastener being removably connectable to the body; or (3) a loop defined by a pair of generally parallel slits in the body of the tube securing device.

### CROSS-REFERENCE TO RELATED APPLICATIONS

This application claims priority to US Application No. 63/763,711 filed on Feb. 26, 2025, the content of which (text, drawings and claims) is incorporated by reference herein.

### STATEMENT REGARDING FEDERALLY SPONSORED RESEARCH OR DEVELOPMENT

Not Applicable.

### BACKGROUND

This application relates generally to devices to hold medical tubing (such as nasal cannulas and nasal gastric (NG) tubes) in place on a patient.

Nasal cannulas, nasal gastric (NG) tubes, IV tubing, and feeding tubes (collectively, medical tubing) are often used to provide patients with medicine, oxygen, or nutrients. It is not uncommon for such medical tubing to be used for extended periods of time (days or weeks, and sometimes longer). The extended use of such medical tubing requires that the medical tubing be secured in place. Typically, this involves taping the tubing in place to the patient. This can pose problems for patients with fragile skin, such as premature babies (“preemies”) and geriatric patients. In such patients, removal of the tape which holds down the tube may damage the skin. This could potentially be resolved using a tape with a weak adhesive. However, such tapes do not maintain the medical tubing in place satisfactorily. In addition, merely taping the medical tubing to the patient leaves the medical tubing against the patient's skin, where the tubing can rub against, and thus irritate, the patient's skin.

It would thus be desirable to provide a device for securing medical tubing in place on a patient which will keep the medical tubing off the patient's skin (at least in the vicinity of the device) and which will reduce the potential for damaging the patient's skin upon removal of the tube-securing device.

### SUMMARY

Described herein is a tube-securing device for securing medical tubing in place on a patient. The tube-securing device comprises a body adapted to be adhered to skin of a patient and has at least one tube securing element adapted to retain medical tubing in place relative to the tube-securing device. The tube-securing device is formed from a material having an adhesive lower surface and a napped upper surface. The at least one tube-securing element comprises one of: (a) a finger having a fixed end and a free end, the finger being pivotal relative to the body about the fixed end; (b) a finger mounted to, and extending from, a fastener, the fastener being removably connectable to the body; or (c) a loop defined by a pair of generally parallel slits in the body of the tube securing device.

In various exemplary embodiments, the tube-securing device is made of a material comprising a top layer, a reinforcing layer, and an adherent layer. The top layer comprises a base material, an upper pile extending from an upper surface of the base material, and a lower pile extending from a lower surface of the base material. The base material is flexible and stretchable, and the upper pile defines the napped upper surface. The reinforcing layer, which in various embodiments is formed from a polyurethane or polyethylene film, is secured to the top layer by a first binder, such as an acrylate binder. The adherent layer comprises an adherent, such as silicone gel, defining the adhesive lower surface. The adherent is operatively bound to the reinforcing layer by a second binder.

In various embodiments, the finger extends from the body such that the fixed end is either at an edge of the body or spaced from the edge of the body, in which case the free end is spaced from or proximate the edge of the body.

In various embodiments, the tube securing device further includes an attachment element adapted to engage the napped surface of the body to maintain the finger in a position such that the finger is folded about the fixed end of the finger to secure the finger against the body. In this way, the adhesive lower surface of the finger faces away from the body and an outer portion of the finger is adapted to fold over in a direction opposite from the fold about the finger fixed end. The finger is thus adapted to define a loop that surrounds the medical tubing.

In various embodiments, the attachment element comprises either a hook-type material or a strip defined by two opposed generally parallel slits in the body. The hook-type material is adapted to engage the napped surface of the base. The strip extends generally perpendicular to at least one finger. In various embodiments, the strip is fixed at its opposite ends, thereby defining a loop through which the finger can extend.

In various embodiments, the tube-securing device includes a nose-surround and at least one cheek portion extending from a right or left side of the nose-surround. In various embodiments, the tube-securing device includes two cheek portions, such that each cheek portion extends from opposite sides of the nose-surround.

In various embodiments, the tube-securing device includes a plurality of tube-securing elements or only one tube-securing element. In various embodiments, the tube-securing device is light transmissive.

Also described herein is an adhesive device for securing medical equipment to the face of a patient. The adhesive device is formed from a material that has an adhesive lower surface and a napped upper surface. The adhesive device includes a nose-surround and a cheek portion extending from each side of the nose-surround. The nose-surround includes a base sized to be positioned between an upper lip and nose of a patient, as well as arms extending from opposite ends of the base, such that the base and arms define an opening sized to fit over the patient's nose.

In various embodiments, the adhesive device is adapted to have medical tubing or a nasal CPAP mask secured to it. In various embodiments, the adhesive device further comprises an upper structure that includes a bridge member extending from an apex of the nose surround. The bridge structure is sized to extend along the bridge of the patient's nose. In various embodiments, the upper structure also includes a cross-bar extending across the top of the bridge member.

Also described herein is a tube-securing device adapted to secure medical tubing to a patient. The tube-securing device includes a base and an attachment element removably securable to the base. The base is formed from a pliable, flexible, conformable material with an adhesive lower surface and a napped upper surface. The attachment element includes an elongate fastener and a finger extending from a first end of the elongate fastener. The elongate fastener has hook elements along the bottom surface of the fastener. The hook elements are adapted to removably engage the napped upper surface of the base to removably hold the attachment element to the base. The finger includes a film with an adhesive layer on an upper surface of the film. The finger has a length such that the finger can be folded over tubing positioned on the fastener and extend along a majority of the length of the fastener.

In various embodiments, the attachment element includes indicia that indicate a position on the attachment element for placement of medical tubing. In various embodiments, the indicia are color-coded such that each color indicates a size of tubing for which the attachment element is designed to be used. In various embodiments, the indicia include a pair of generally parallel spaced apart lines extending generally perpendicular to an axis of the attachment element.

### BRIEF DESCRIPTION OF THE DRAWINGS

FIG. 1 is a schematic cross-sectional view of the material from which the tube-securing devices are made;

FIG. 2 is a plan view of a first tube-securing device with medical tubing positioned on top of the tube-securing device;

FIG. 3 is a plan view of the device demonstrating a first step in using the first tube-securing device;

FIG. 4 is a plan view of the device demonstrating a second step in using the first tube-securing device;

FIG. 5 is a schematic cross-sectional view taken along line A-A of FIG. 4 showing the medical tubing secured in place by the first tube-securing device with a release liner still in place on the tube-securing device;

FIG. 6 is a plan view of a tube securing device adapted to secure a nasal CPAP mask to a patient;

FIG. 7 is a plan view of a second tube-securing device;

FIGS. 8 and 9 are plan views showing the steps of using the second tube-securing device to secure medical tubing;

FIG. 10A is a plan view of a third tube-securing device ready for receiving tubing;

FIG. 10B is a plan view of the third tube-securing device with tubing secured in place;

FIG. 11 is an enlarged fragmentary view of the third tube-securing device taken at the circle C of FIG. 10A;

FIG. 11A is a cross-sectional view taken along line B-B of FIG. 10B;

FIG. 11B is a schematic cross-sectional view taken along lines C-C of FIGS. 10B and 11A;

FIGS. 12A-B show steps of an alternate method of securing a tube to the third tube-securing device of FIG. 10A;

FIG. 12C is a cross-sectional view taken along line D-D of FIG. 12B;

FIG. 12D is a schematic cross-sectional view taken perpendicularly to the line D-D of FIG. 12B;

FIG. 13 is a plan view of a fifth tube-securing device;

FIGS. 14 and 15 are alternate schematic cross-sectional views taken along line E-E of FIG. 13;

FIG. 16 is a schematic plan view of a fifth tube-securing device;

FIGS. 17A-B are a schematic plan view and a schematic cross-sectional view showing a first method of using the fifth tube-securing device of FIG. 16, with the cross-section of FIG. 17B being taken along line F-F of FIG. 17A;

FIG. 18A is a schematic plan view of a variation of the tube-securing device of FIG. 16;

FIGS. 18B-C are a schematic plan view and a cross-sectional views, respectively, of the tube-securing device of FIG. 18A, with FIG. 18C being taken along line G-G of FIG. 18B showing a method of using the tube-securing-device of FIG. 18A

FIGS. 19A-B are cross-sectional views of further tube-securing device showing a tube ready to be secured to the tube-securing device (FIG. 19A) and the tube being secured in place (FIG. 19B);

FIG. 20A comprises plan view of three strip-type tube-securing devices sized which are coded to secure medical tubing of different sizes;

FIGS. 20B-D are schematic cross-sectional views showing the steps of use of the securing devices of FIG. 20A;

FIG. 21 is a plan view of a nasal tube-securing device;

FIG. 22 is a plan view of a tube-securing device designed for use with gastronomy/jejunostomy tubes;

FIGS. 23 and 24 are views of a sixth tube-securing device shown adhered to a doll;

FIGS. 25A-B show a tube-securing device wherein the tubing itself includes a hook-type material (such as Velcro);

FIG. 26A and B are front and back plan views of a seventh tube-securing device;

FIG. 26C is a back plan view of the seventh tube-securing device with the release liner removed from a cheek portion of the tube-securing device; and

FIGS. 27A-E show the steps of applying the seventh tube-securing device to a patient (as demonstrated on a baby-doll).

Corresponding reference numerals will be used throughout the several figures of the drawings.

### DETAILED DESCRIPTION

The following detailed description illustrates the claimed invention by way of example and not by way of limitation. This description will clearly enable one skilled in the art to make and use the claimed invention, and describes several embodiments, adaptations, variations, alternatives and uses of the claimed invention, including what we presently believe is the best mode of carrying out the claimed invention. Additionally, it is to be understood that the claimed invention is not limited in its application to the details of construction and the arrangements of components set forth in the following description or illustrated in the drawings. The claimed invention is capable of other embodiments and of being practiced or being carried out in various ways. Also, it is to be understood that the phraseology and terminology used herein is for the purpose of description and should not be regarded as limiting.

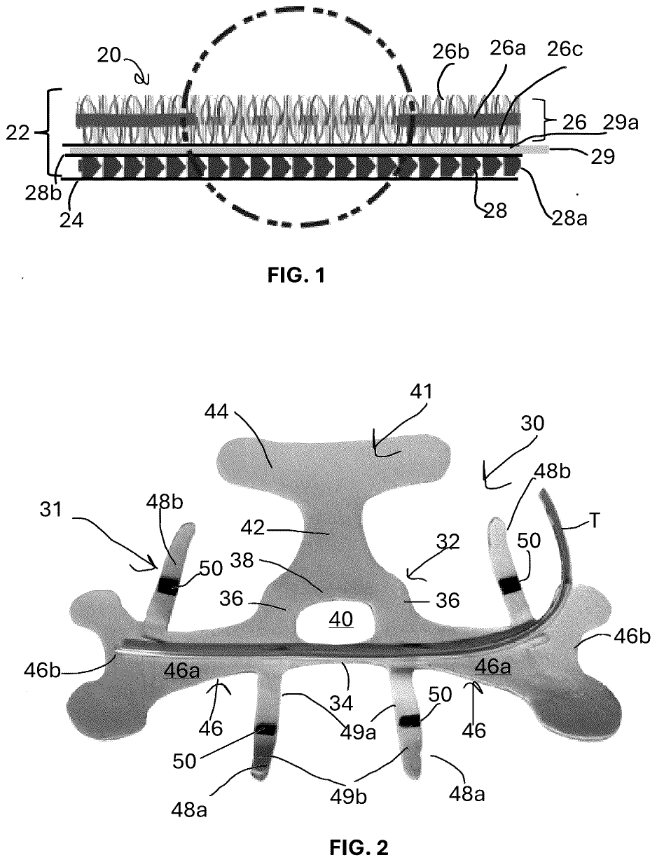

A tube-securing device 30 is shown in FIG. 2 with medical tubing T on top of the device. Initially, the device is made from an adhesive material 20 (shown schematically in FIG. 1). The adhesive material 20 is substantially the same material as disclosed in WO2024226615, which is incorporated herein by reference. The adhesive material 20 is an adhesive assembly comprising an elastic, stretchable and flexible barrier material 22 and a release liner 24. The barrier material 22 comprises a top layer 26, a reinforcing layer 29, and an adherent layer 28. The release liner 24 is removably secured to the adherent layer 28 of the barrier material 22 to protect the adherent layer 28 until the barrier material is put into use. When the release liner 24 is removed, the adherent layer 28 will removably adhere the device to a surface, such as a patient's skin.

The top layer 26 preferably comprises a stretchable, elastic base material 26a which can be formed from a knit material, a woven material, or a tricot material. Preferably the top layer 26 is elastic and stretchable in all directions, rather than in just one direction. The material which forms the base material 26a can comprise man-made fibers, natural fibers, or a combination thereof. Preferably, the base material 26a is formed from a combination of fibers, at least a portion of which are elastic fibers, such as spandex (a polyether-polyurea copolymer), such as, for example, Lycra®. If formed from a combination of fibers, the elastic fibers can be combined, intertwined, or formed with, for example, natural fibers, such as cotton fibers, or man-made fibers, such as nylon or spandex, to form the thread or yarn from which the material for the top layer 26 is formed. The elastic fibers provide the stretchability and elasticity of the top layer 26. The base material can, for example, be about 5% to about 95% nylon and about 95% to about 5% spandex. In a preferred embodiment, the base material is about 90% nylon and about 10% spandex. Illustratively, the nylon can be SD Nylon and the spandex can be 40 Denier Spandex. Further, the material can comprise different types of nylon, such as “50/17 SD” nylon and “20/1 SD” nylon. The two types of nylon can be combined in ratios of about 4:1 “50/17 SD” Nylon:“20/1 SD” Nylon to about 1:4 “50/17 SD” Nylon:“20/1 SD” Nylon, and preferably about 3.2:1 “50/17 SD” Nylon:“20/1 SD” Nylon. In a preferred embodiment, the material comprises about 69% “50/17 SD” nylon, about 21% “20/1 SD” nylon, and about 10% 40 denier spandex. Further, the base material 26a is composed of materials which are generally inert and generally biocompatible. Fibers other than Nylon and spandex can be used. For example, the stretchable (spandex) component of the flexible material could comprise silicone and/or natural rubber; and the nylon could be replaced with polyethylene and/or polyurethane. The base material can be made of other types of natural fibers or man-made/industry-made fibers (or combinations thereof). For example, the other man-made/industry-made fibers can include carbon fibers, expanded polytetrafluoroethylene (commonly known by the brand name, Gore-Tex®), or other synthetic and/or conductive fibers. The top layer 26 can be made, as noted above, by weaving or knitting the fabric top layer. If the top layer is made from man-made materials, the top layer can be formed by 3D printing.

The top layer 26 further comprises an upper pile 26b and a lower pile 26c which extend from the upper and lower surfaces, respectively, of the base material 26a to define upper and lower napped or plush surfaces 26b,c of the top layer 26. The upper and lower piles 26b,c comprise fibers, pile, or napping. It is noted that “pile”, “plush” and “nap” are used synonymous and are used interchangeably herein.

The upper and lower pile 26b,c can be formed by brushing or abrading the surfaces of the base material 26a to create fibers, pile, or napping on each side of the base material. As such, the upper and lower pile 26b,c have the same composition as the base material 26a. The upper and lower pile 26b,c, depending on how they are formed, can be straight, curved, twisted, etc. Further, the fibers/pile/napping may not be uniformly shaped. The lower pile 26c can have fibers extending from the bottom of the base material 26a that greatly increase the surface area of the bottom of the top layer 26 as compared to a “smooth” (i.e., un-napped) material.

The upper and lower piles 26b,c can be formed by brushing the base material 26a in a single direction (preferably in a machine direction of the material). We have found that when the pile/napping is formed in this manner, when the barrier material 22 is folded over on itself along a line generally parallel to the direction of brushing, such that upper pile contacts upper pile, the two layers of material can move relative to each other in a direction generally parallel to the fold relatively freely, and movement in this direction (generally perpendicular to the direction of brushing) is like the fabric is moving along track. On the other hand, when the fabric layers are moved relative to each other in the machine direction (i.e., parallel to the direction of brushing), when the fabric is moved in the same direction as the brushing was performed, the fabric moves freely. However, when moved against each other in a direction opposite to the direction of brushing, there is some friction, like the material is grabbing at itself.

A reinforcing layer 29 is positioned below the top layer 26, and is adhered to the top layer by an adhesive first binder 29a, such as an acrylate binder, applied to the top side of the reinforcing layer. This reinforcing layer 29 can be made from a polyurethane film or a polyethylene film and can add some slight rigidity to the barrier material 22, which will make the material less “floppy” and a bit easier to handle, especially when wearing gloves. Although the reinforcing agent will add some rigidity to the barrier material 22, the reinforcing layer is stretchable, and, preferably, elastic, in all directions, such that use of the reinforcing layer 29 will not diminish the stretchability or elasticity of the top layer 26. If a binder 29a is used, the binder can be, for example, an acrylic or acrylate adhesive which will bond to the reinforcing layer 29 and which will secure the reinforcing layer 29 to the top layer 26. The first binder 29a which adheres the reinforcing layer 29 to the top layer 26 extends into the lower pile 26c (or the lower pile 26c extends into the binder) to increase the contact surface area of the upper layer with the binder. The binder also bonds with the reinforcing layer, such that the reinforcing layer cannot easily be removed from the top layer 26.

The adherent layer 28 is secured to the bottom surface of the reinforcing layer 29. As disclosed in US20230046036, which is incorporated herein by reference, the adherent layer 28 comprises at least an adherent 28a which adheres to, but does not aggressively bond to, skin and is gentle on the skin during long term wear as well as during removal. Thus, the barrier material 22 can be removably attached to a patient's skin, and damage to the skin during removal of the barrier material 22 is minimized by the use of the adherent 28a. The adherent 28a preferably comprises a silicone gel material or a hydrogel adhesive, and has viscoelastic and/or high co-adhesive properties. It can be applied to the bottom surface of the reinforcing layer 29 at a heavy (high) coat weight thickness of about 100-500 g/m², and preferably about 250-300 g/m₂. Such adhesives are typically composed of a very lightly cross-linked silicone elastomer whose polymer network has been swollen with silicone fluids. The silicone gel has elastic characteristics, is stretchable in any direction, and will elongate with the top layer 26 and reinforcing layer 29. That is, as the silicone gel elongates, the silicone gel layer becomes thinner. The thinning of the silicone gel layer is constant, and thus, the silicone gel layer does not get thinner in just one area, but rather thins uniformly. Thus, the silicone gel layer is colinear. The same is true for the top layer 26 and the release liner 24.

Preferably, the adherent layer 28 additionally includes a second binder 28b that secures the silicone gel adhesive to the bottom surface of the reinforcing layer 29. The second binder 28b is preferably the same as the first binder 29a. The binder bonds to both the adherent 28a and the reinforcing layer 29 to securely fix the adhesive layer to the reinforcing layer. If the second binder 28b is used, the binder is covered by the adherent 28a, such that the second binder 28b, which in various embodiments can be an acrylate adhesive, will not come into contact with the patient's skin during use. The reinforcing layer 29 is preferably coextensive with at least the colinear adherent layer 28, and more preferably coextensive with the top layer 26. So that damage to the patient's skin is reduced or minimized upon removal of the device 10, the adherent 28a has an adhesive strength sufficiently low so as to substantially prevent trauma to human skin when the device is removed from the patient's skin, i.e., the higher the adhesive strength, the higher the surface tension and, thus, the higher the probability of causing tissue damage. As described in our US Pub Nos. 20180110659 and 20180008477, which are incorporated herein by reference, the adherent 28a preferably has a strength of about 10 oz/in to about 50 oz/in.

The release liner 24, as noted, is removably secured to the adherent layer 28. The release liner 24 is preferably made from a generally non-stretchable material, which is held to the adherent layer 28 by the natural adhesive qualities of the silicone adherent. The adhesive force of the adherent layer 28 is substantially less than the adhesive force of the second binder 28b, and thus, removal of the release liner 24 from the adherent layer 28 will not pull the adherent layer 28 from the top layer 26 or the reinforcing layer 29. The material for the release liner 24 is chosen by design to be a material which has low adhesion force with respect to the silicone adherent. The release liner 24 does not directly contact the top layer 26 (or reinforcing layer 29). Thus, the release liner 24 is removably adhered to the bottom of the barrier material 22 by the silicone adherent layer 28. The release liner 24 could have a coating, for example, of fluorosilicone applied to the side of the release liner which is in contact with the silicone gel adherent. This will facilitate removal of the release liner 24 from the barrier material 22. Additionally, the release liner could extend beyond the perimeter of the barrier material construction 22 for ease of removal and then storing of a device when not in use. In an alternative, the device 10 could be provided with tabs or perforations, as disclosed, for example, in U.S. Pat. No. 9,427,222 , which is incorporated herein by reference, to facilitate removal of the release liner 24 from the barrier material 22.

Turning to FIG. 2, the tube-securing device 30 is adapted for use to secure medical tubing, such as a nasal cannula, on a patient. As is known, a nasal cannula has two outputs sized to be received in or just below a patient's nostrils to deliver oxygen to a patient through cannula outputs. As can be appreciated, the upper pile 26b of the material forms the upper surface of the device 30, and the release liner 24 forms a lower surface of the (as provided) device. As discussed below, the device is applied to a patient by removing the release liner to expose the adhesive so that the device 30 can be applied to a patient. As applied, the upper surface of the barrier material 22, and hence the upper pile 26a, will face away from the patient.

The device 30 includes a body 31 comprising a nose surround 32 and cheek portions 46. The nose surround 32 comprises a base 34 sized to be positioned under a patient's nose and above the patient's lip. Preferably the base 34 is sized such that when positioned under the nose, it will not extend onto the patient's upper lip. A pair of arms 36 extend upwardly from the opposite ends of the base 34. The arms 36 are sized to extend along the opposite sides of the patient's nose to approximately the bridge of the nose. A top 38 extends between the upper ends of the arms 36 and is sized to pass over the lower end of the bridge of the nose. The nose surround 32, as seen in FIG. 2, defines an opening 40 which is sized to fit over a patient's nose. The device 30 can include an optional upper structure 41 comprising bridge member 42 and a cross-bar 44. The bridge member 42 extends upwardly from the middle of the body top 38 and is sized to extend along the bridge of the patient's nose. It is preferably sufficiently long to extend to a point between the patient's eyebrows. The cross-bar 44 extends across the top of the bridge member 42. As seen, the upper structure 41 is generally T-shaped. The bridge member 42 has a length sufficient that the cross-bar 44 will extend outwardly from the patient's bridge above the patient's eyebrows.

A cheek portion 46 extends outwardly from each of the arms 36, such that there is a cheek portion on both the left and right side of the nose surround 32. The cheek portions each include a generally rectangular portion 46a and an optional enlarged end portion 46b at the end of the rectangular portion 46a. A first pair of securing fingers 48a extend downwardly below the nose surround arms 36; and a second pair of securing fingers 48b extend upwardly from the cheek portions 46, preferably closer to the end of the rectangular portion 46a. The fingers 48a,b each include an attachment element 50 positioned between the bases and free end of the fingers. The attachment element 50 thus divides the fingers 48a,b into an inner portion 49a extending from a base of the finger to the attachment element 50 and an outer portion 49b extending from the attachment element 50 to the free end of the finger. The attachment element 50 is adhered to the upper pile 26b of the barrier material 22 which forms the device 30 and is adapted to removably adhere to the upper pile 26b. The attachment device 50, for example, can be the hook portion of hook and pile (i.e., Velcro®) fastener. The hook portion of the attachment element 50 is upwardly facing (with reference to FIG. 2).

As can be appreciated, the device 30 can be formed in multiple sizes to accommodate patients of different size, from premature infants through adults. In addition to having a low adhesive strength, the device 30 can be light transmissive, such that light can pass through the device. This can be beneficial in premature infants who are jaundiced and require light therapy to treat the jaundice.

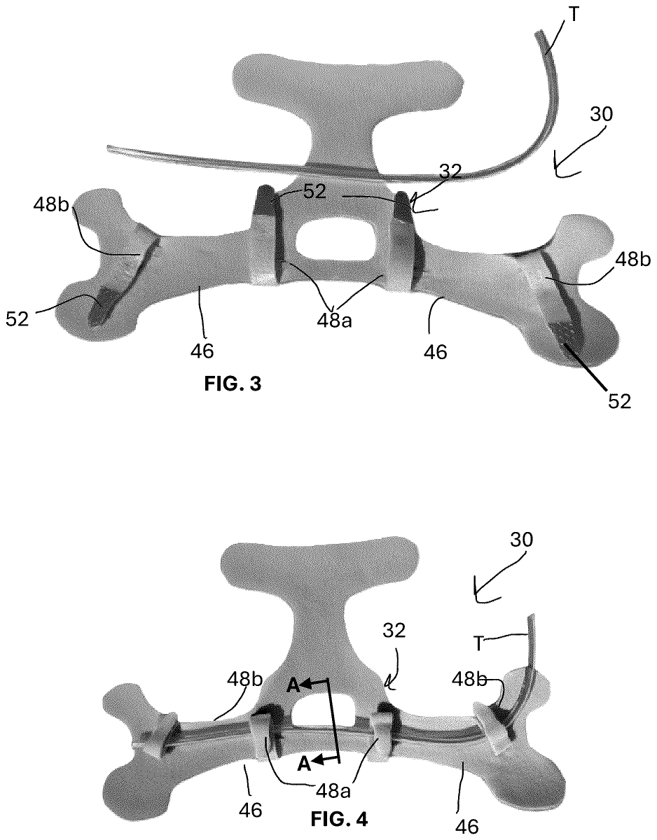

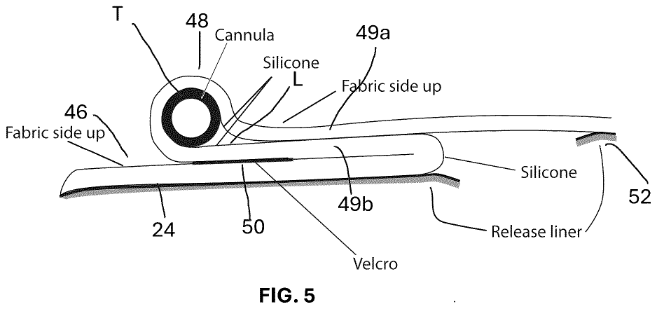

Use of the device 30 is shown in FIGS. 3-5. Initially, the device 30 is positioned on the patient's face, with the nose-surround 32 being about the patient's nose and the cheek portions 46 extending over the patient's cheeks in a smooth manner. To apply the device 30 to the patient, the release liner 24 is removed from the back of the device 30. Preferably, the release liner 24 is sliced or slit so that, when the release liner is removed from the device, the release liner 52 at the ends of the fingers 48a,b remains with the fingers, as seen in FIG. 5. The release liner (other than the portions 52 at the ends of the fingers) can be removed all at once, or the release liner can be removed in halves (i.e., first the right half and then the left half). Leaving a portion of release liner at the ends of the fingers provides an area of the fingers 48a,b that can be grasped without contacting adhesive. Removal of the release liner exposes the silicone adhesive on the back of the nose surround 32, the cheek portions 46, the (optional) upper structure 41, and the majority of the fingers 48a,b. The device 30 can be adhered to the patient by pressing lightly against the device as it is positioned on the patient's face. In this manner, the entire device, except for the fingers 48a,b will be adhered to the patient.

A first step of securing the tubing T, shown in FIG. 3, comprises folding the fingers 48 over at their fixed ends or bases, such that the attachment element 50 of each finger engages the upper pile 26b of the cheek portions 46. By lightly pressing against fingers, the attachment elements 50 will hold their respective fingers 48a,b in the folded over position shown in FIG. 3. In this position, the silicone adhesive of the fingers will be facing upwardly (or away from the patient). The inner portion 49a of the fingers 48a,b will be fixed in place, but the outer ends 49b of the fingers will still be free.

Next, the tubing T is positioned on the patient with the nasal cannula output posts (not shown in the figures) aligned with the patient's nostrils. The tubing T is then laid over the outer portion 49b of the fingers 48a,b inwardly of the free ends of the fingers. The outer portion 49b of the fingers 48a,b are then folded over again, as shown in FIG. 4, to encompass or surround the tubing T, as seen in cross-section of FIG. 5. In this step, the attachment elements 50 define a fold line for each finger. In this position, the silicone adhesive of the upper portion 49b of each finger will be in contact with the tubing (which is generally made of silicone) and with the adhesive (i.e., silicone) of the inner portion 49a of the finger. Thus, the fingers 48a,b, as folded over in FIG. 5 forms a loop L which surrounds and retains the tubing T. Because the adhesive of the two portions 49a,b of the fingers will be in contact, it will be difficult for the tubing T to be inadvertently pulled through out of the loop L. The adhesive of the fingers 48a,b will, as noted, be in contact with the tubing T. The adhesive will thus adhere to the tubing to substantially prevent inadvertent movement of the tubing relative to the patient. This will enhance the patient's comfort. Further, as seen, the tubing T, at least in the area of the tube-securing device, will lie on the tube-securing device 30, rather than on the patient's skin. This will further increase the patient's comfort when having a nasal cannula.

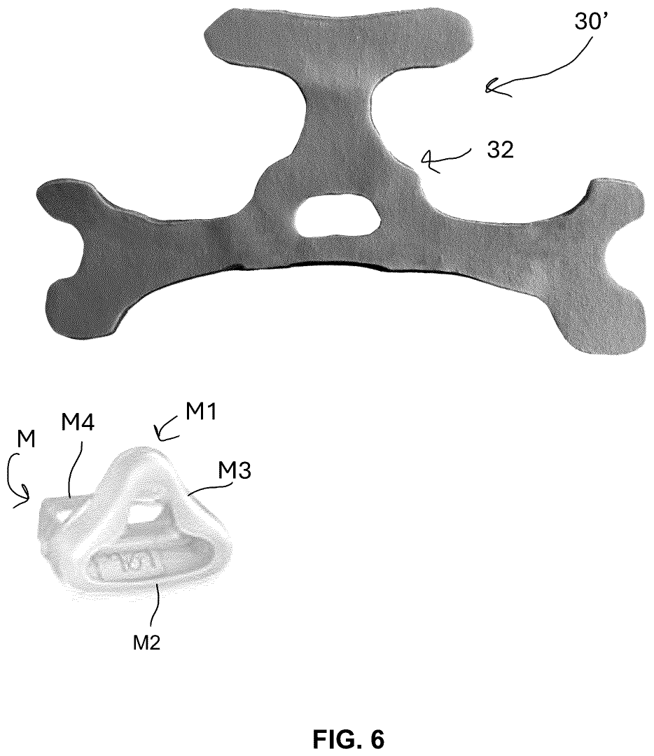

FIG. 6 shows an device 30′ which is identical to the tube-securing device 30, except for the fact that the device 30′ does not include the fingers 48a,b of the device 30. The device 30′ thus need not be described in detail. In the device 30′, a nasal Constant Positive Airway Pressure (CPAP) mask M is adhered to the nose surround 32 of the device 30′. As is known, nasal CPAP masks include a nose surround M1 which, as seen in FIG. 6 is generally triangular in shape. The nose surround includes a base M2 which passes beneath the user's nostrils and two side members M3 which extend from the ends of the base M2 and meet at an apex. A tube mount M4 extends outwardly from the base M2 of the nose surround M1. To secure the CPAP mask M to the device 30′, hook material (such as Velcro hook material) can be fixed to the arms M3 of the CPAP mask. The hook material will then engage the upper pile 26b (FIG. 1) of the device 30′ to hold the CPAP mask M to the device 30′. The hold of the hook material to the device 30′ will be sufficient to maintain the CPAP mask to the device during use. As can be appreciated, the use of the device 30′ may be more comfortable than the straps typically used to hold the nasal CPAP mask in place.

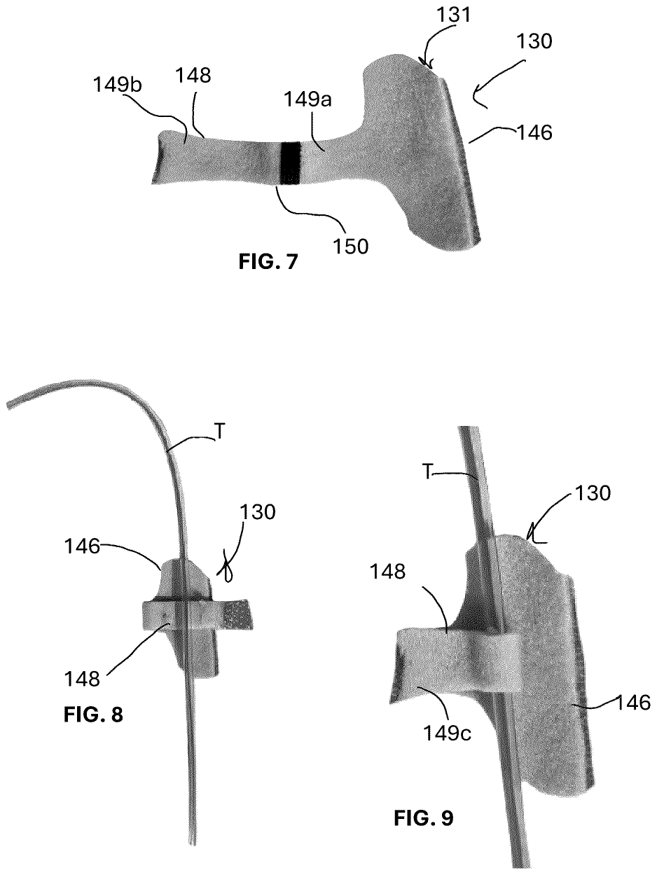

FIG. 7 shows a second embodiment of the medical tube-securing device 130 which, like the tube-securing device 30, is made from the adhesive material 20. The tube-securing device 130 includes a body 131 comprising a cheek portion 146 which is generally rectangular in shape, but can be any desired shape. Preferably, the cheek portion 146 has width that is no larger than the patient's cheek. A single finger 148 extends from the approximate center of the cheek portion 146 and includes an attachment element 150 (which, for example, can be Velcro hook material or an adhesive) spaced from the base of the finger 148 which divides the finger into inner and outer portions 149a,b, respectively. Use of the tube-securing device 130, as seen in FIGS. 8-9, is generally identical to that of the device 30. However, the device 130 can be used, for example, to hold an NG tube in place against a patient's face. As seen in FIG. 9, the tube T will extend along the elongate cheek portion 146, such that the tube will not contact the patient's skin in the area of the tube-securing device. Although the attachment element 150 is shown to be positioned midway along the length of the finger 148, the attachment element could be positioned closer to the base of the finger (i.e., where the finger joins the body). This would shorten the inner portion 149a of the finger and lengthen the outer portion 149b of the finger. By lengthening the outer portion 149b of the finger, the excess material 149c that extends beyond the tubing T after the outer portion 149b has been wrapped about the tubing is lengthened. There is thus a longer “tail” that can be applied to the patient's skin to enhance the ability of the device 130 to hold the tubing T in place relative to the patient.

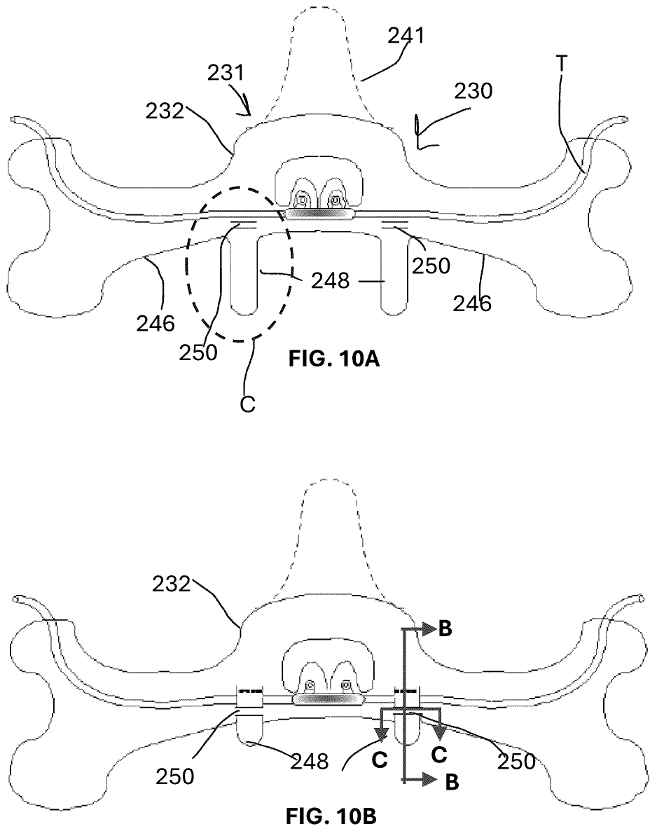

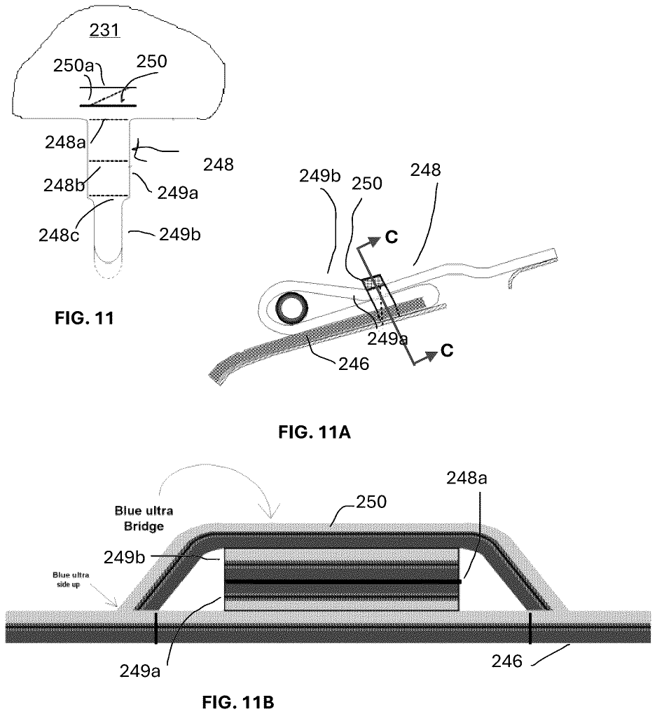

FIGS. 10A-12D show a third tube-securing device 230. As with the devices 30 and 130, the tube-securing device 230 comprises a body 231 comprising a nose-surround 232 and cheek portions 246 extending from opposite sides of the nose surround 232. A single pair of fingers 248 extends downwardly from beneath the base of the cheek portions 246. As seen in FIG. 11, the fingers 248 can be formed with a base or inner portion 249a and a free or outer portion 249b which is narrower than the inner portion 249a. An optional upper structure 241 extends upwardly from the top of the nose-surround 232, and is sized to cover the bridge of a patient's nose. Rather than providing the fingers 248 with an attachment element in the form a hook portion of a hook and loop fastener, the device 230 is provided with a pair of slits 250a in each cheek portion 246 above the base of each finger 248 which define a bridge or loop 250 (akin to a belt loop) which then functions as the attachment member.

The slits 250a are through-slits, and, with reference to FIG. 1, extend at least through the barrier material 22. The slits 250a can also extend through the release liner 24. Additionally, the device 230 includes a slit 248a at the base of the finger 248, a slit 248b approximately midway along the length of the finger inner portion 249a, and a slit 248c at the junction between the inner and outer portions 249a,b of the finger 248. The slits 248a-c are all back-slits and extend only through the release liner. Although not shown, the device can also include back slits extending between the opposite ends of the slits 251a which define the bridge 250.

In use, the release liner is removed from the body 231 of the device 230. This will expose the adhesive on the body 231 and nose surround 241 of the device and the device can be applied to a patient's face. However, due to the various slits just noted, the release liner will remain with the fingers 248. With the device 230 applied to the patient's face, starting from the initial state shown in FIG. 10, the fingers 248 are folded about their base and slid beneath the loop 250. This will result in the adhesive side of the fingers facing upwardly facing the adhesive of the bridge. However, because the release liner initially remained with the finger, the adhesive of the finger did not contact the adhesive of the bridge while the finger was passed through the bridge, and thus the finger 248 is able to be passed through the bridge without too much difficulty. At this point, the release liner in the base between cuts 248a and 248b is removed. The tubing T is then placed over the base of the fingers above the loops 250 to be in contact with the now-exposed adhesive of the base portion. The outer portion 249b of the fingers are then folded about the tube and passed through the loops 250 a second time. During this process, the release liner in the second half of the base between cuts 248b and 248c is removed. This will form two layers of the finger that pass through the loop, as seen in FIG. 11B, with the adhesive of the outer portion 249b contacting the adhesive of the inner portion 249a of the fingers. Further, the tubing T will be in contact with the adhesive of the finger 248 where the finger wraps about the tubing T. This will securely hold the tubing T in place relative to the device 230 and will substantially prevent movement of the tubing T relative to the device 230. The slits 250a and the loop or bridge 250 are long enough so that the bridge can be lifted out of the plane of the device 230 to pass a finger 248 beneath the bridge twice.

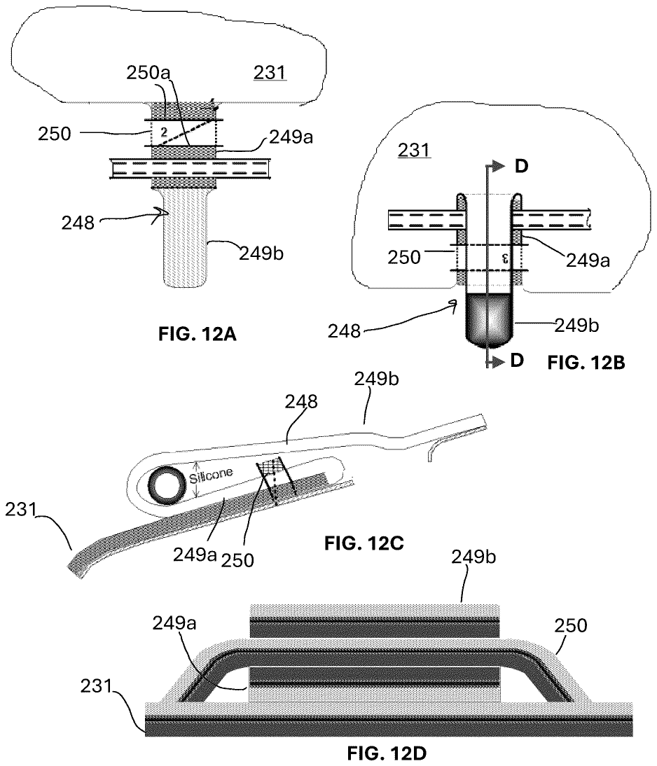

An alternate method of use is shown in FIGS. 12A-C. In this method, the initial steps are the same as just described. However, rather than passing the finger 248 beneath the bridge 250 a second time, when the finger 248 is wrapped about the tubing T, the free end 249b of the finger is positioned over the bridge, as seen in FIGS. 12C and 12D. The free end 249b of the finger is then adhered to the patient. In this case, the adhesive of the finger 248 will engage the top of the bridge, as seen in FIG. 12D. This method of application may be somewhat easier than the method of application described with respect to FIGS. 11A-B.

The loop 250 could alternatively be defined by two strips of material which extend away from each other, and which can be flexed to surround the tubing and engage each other. These two ends would effectively define a belt, the ends of which can be joined together to hold the finger in its loop defining configuration. This alternative version would avoid the need to thread the finger through the loop 250.

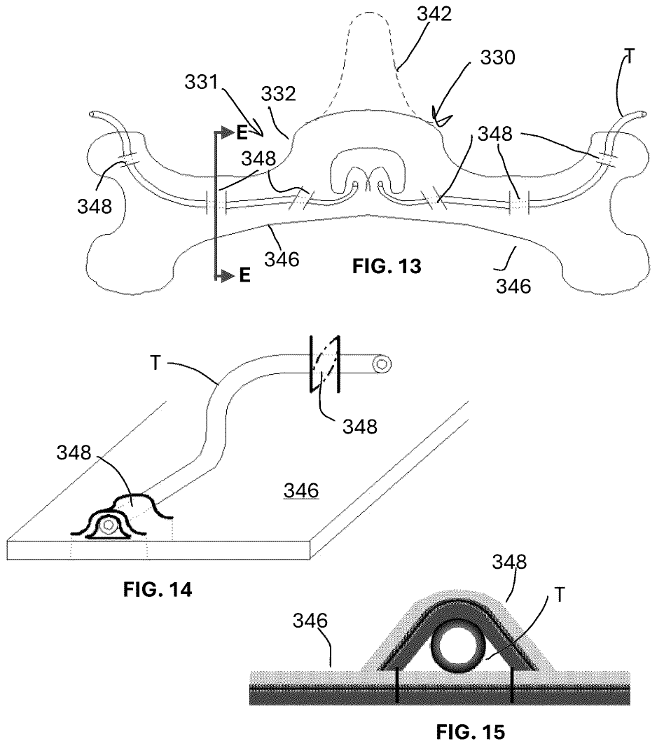

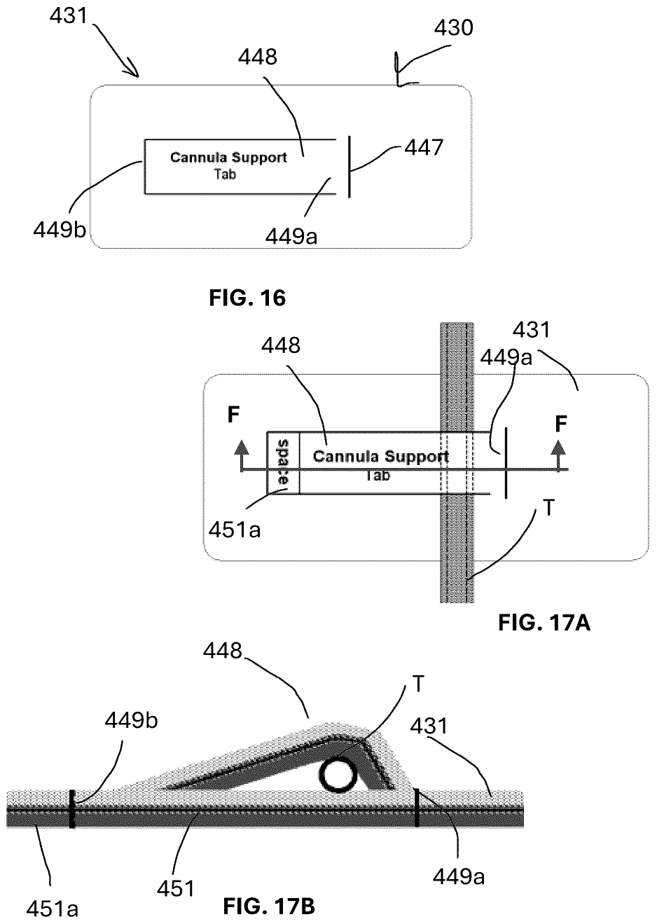

FIGS. 13-15 show a fourth tube-securing device 330. As with the devices 30, 130, and 230, the tube-securing device 330 includes a body 331 comprising a nose-surround 332 and cheek portions 346 extending from opposite sides of the nose surround 332. An optional upper structure 342 extends upwardly from the top of the nose-surround 332. Unlike the prior versions, the device 330 does not include fingers. Instead, it is provided only with a series of loops 348 defined by spaced apart slits through which the tubing T can be threaded. The tubing T is thus held in place by the loops 348. As seen in FIG. 15, the silicone adhesive of the loop 348 is in contact with the tubing T to substantially prevent inadvertent axial movement of the tubing T relative to the device 330. Inadvertent movement of the tubing T is also substantially reduced by the number of loops 348 in the device. As seen, there are three loops 348 per side, which will create a frictional grip on the tubing.

A fifth tube-securing device 430 is shown in FIG. 15. The tube-securing device 430 comprises body 431 into which at least one finger 448 is formed. The finger is formed by a pair of parallel slits which extend from a base or fixed end 449a and a free end 449b of the finger 448. The fixed end 449a of the finger 448 is spaced from an edge of the body 431. The free end 449b is also shown spaced from the edge of the body (so that the finger is fully surrounded by the body 431. In this instance, an end cut extends between the ends of the two parallel cuts so that the free end 449b of the finger 448 can be raised relative to the body 431. Alternatively, the free end of the finger can be at the edge of the body 431 (or even extend beyond the edge of the body). In this case, because the parallel cuts which form the finger 448 will extend inwardly from the edge of the body, there is no need for the end cut which defines the free end of the finger. An optional slit 447 which extends through the thickness of the material is formed behind the fixed end 449a of the finger. That is, the slit 447 is spaced from the inner ends of the two parallel cuts defining the finger 448.

A first method of use of the tube-securing device 430 is shown in FIGS. 17A-B. In this method, after the tube-securing device is adhered to the patient the finger 448, the finger 448 is raised from the plane of the body 431. As can be appreciated, this will form a slot in the body 431 which exposes the patient's skin. The tubing is placed across the slot of the tube-securing device body formed by the raised finger proximate the base or fixed end 449a of the finger to extend across the slot and beneath the finger. Preferably, the tubing T will be generally perpendicular to the slot and the finger 448. The finger 448 is then laid back down into the slot over the tubing T, as shown in FIG. 17B, to re-adhere the finger 448 to the skin. As seen in FIG. 17A, B, there is a short extent 451a of the slot 451 that remains open when the finger is laid back down and applied to the patient's skin. In this method, the slit 447 in the body is not used, and can be omitted.

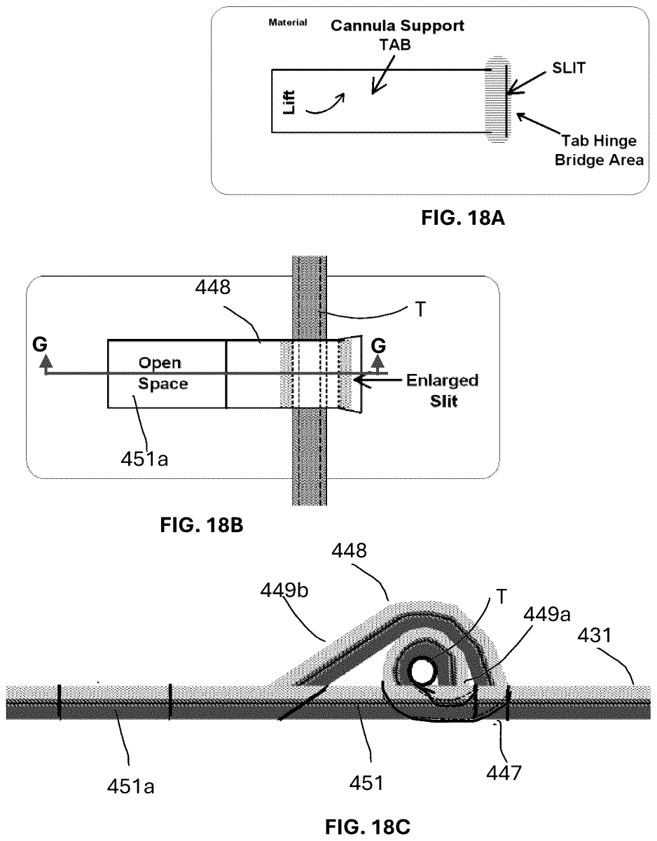

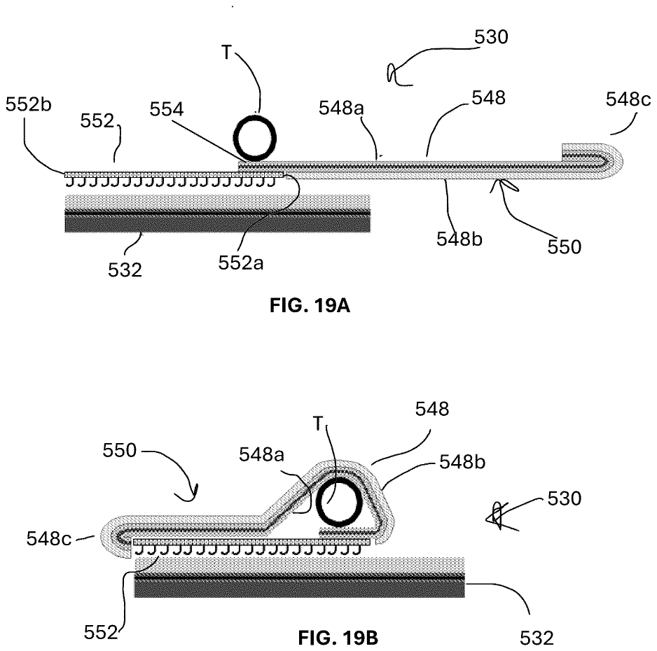

A second method of use of the tube-securing device 430 is shown in FIGS. 18A-B. In this method, after the tube-securing device 431 is adhered to the patient, the finger 448 is raised from the plane of the body 431, forming the above noted slot in the body 431 which exposes the patient's skin. The tubing is placed across the slot of the tube-securing device body formed by the raised finger proximate the base or fixed end 449a of the finger to, preferably, be generally perpendicular to the slot. In this method, though, the finger 448 is initially wrapped (counterclockwise with reference to FIGS. 18B-c) about the tube T. The free end of the finger 448 is then passed upwardly through the slit 447, extended over the portion of the finger overlying the tube T (to form a double layer of the material) and then laid back down into the slot, as shown in FIG. 18B, to re-adhere the finger 448 to the skin. In this method, the finger will fully surround the tubing, substantially eliminating any potential for the tubing to be moved along the length of the slot. In this second method, the tube T is preferably wrapped by the finger prior to applying the tube-securing device 430 to the patient. This will make it easier to secure the tube T to the tube-securing device.

In either instance, the release liner can be cut, so that when the release liner is removed from the body 431, the release liner stays with the finger 448. This will make lifting the finger 448 relative to the body easier. When the finger is raised, the release liner in the finger can be removed.

As can be appreciated, in the foregoing embodiments, the fingers 48a,b, 148, and 248a, 448, and the loops 348 define tube-securing elements for the tube-securing devices.

FIGS. 19A-B schematically show a further tube-securing device 530 which, as will become apparent, can be formed in strips. The device 530 comprises a base 532 formed from the adhesive material 20. The base 532 thus has an adhesive lower surface (protected by a release liner) and a plush, piled, or napped upper surface. The base 532 is preferably formed as a generally rectangular strip, but could have any desired shape. An attachment element 550, in the form of a mounting strip, is removably adherable to the base. The mounting strip 550 comprises a fastener 552 and a finger or extension 548. The fastener 552 is formed from a hook-type material, such as the hook portion of Velcro. The fastener is preferably formed as a rectangular strip having a first end 552a and a second end 552b. Although preferably formed as a rectangular strip, the fastener could have any desired shape. The hooks of the fastener can engage the plush, pile, or napping of the upper surface of the base 532 to be removably connected to the base. The extension (or finger) 548 extends from one first end 552a of the fastener 552. The extension 548 is preferably formed from a film having an adhesive surface 548a and a back surface 548b. As seen in FIG. 19A, an end of the back surface 548b abuts an end of the fastener 552, and the adhesive upper surface extends over (or overlaps) the top surface of the fastener 552 to form an overlap portion 554. This overlap portion 554 of the extension 548 can be adhered to the upper surface of the fastener 552 in any known manner. The free end 548c is folded over upon itself. The extension 548 has a length from its fixed end (at the overlap portion 554) to its free end 548c that is longer than the length of the fastener 552.

In use, the base 532 is applied to the patient. The attachment element or mounting strip 550 can be pre-applied to the base 532, by pressing the hook side of the fastener 552 into the pile of the base. Alternatively, the attachment element can be applied to the base after the base has been applied to the patient. Tubing T is positioned to extend across the attachment portion, preferably at the overlap area 554 and in a direction that is preferably generally perpendicular to the axis of the extension or finger 548. The extension 548 is then folded over the tubing, as seen in FIG. 19B, so that the adhesive of the upper surface 548a contacts the tubing T. Additionally, by positioning the tubing at the overlap area the upper (or adhesive) portion of the extension can then be adhered to the upper surface of the fastener to provide an area of extended attachment of the extension or finger 548 to the fastener 552. As seen in FIG. 19B, the finger 548 is sized so that the turned end of the finger will abut the second end 552b of the fastener.

The device 530 has a few advantages. Firstly, it is not limited to use on a patient's face, but could be applied on a patient's arm, leg, torso, or wherever it might be necessary to secure medical tubing in place relative to the patient. Secondly, because the attachment element 550 is removable from the base 532, if tubing needs to be replaced, the attachment element 550 can be lifted off the base 532 with the tubing, and a new attachment element 550 can be mounted to the base. In this instance, it can be seen that the free end 548c of the finger can form a grasping area that may facilitate removing the attachment element from the base. The ability to leave the base 532 in place on the patient when tubing is replaced avoids the need to pull an adhesive device from the patient's skin, and thus is more protective of the patient's skin.

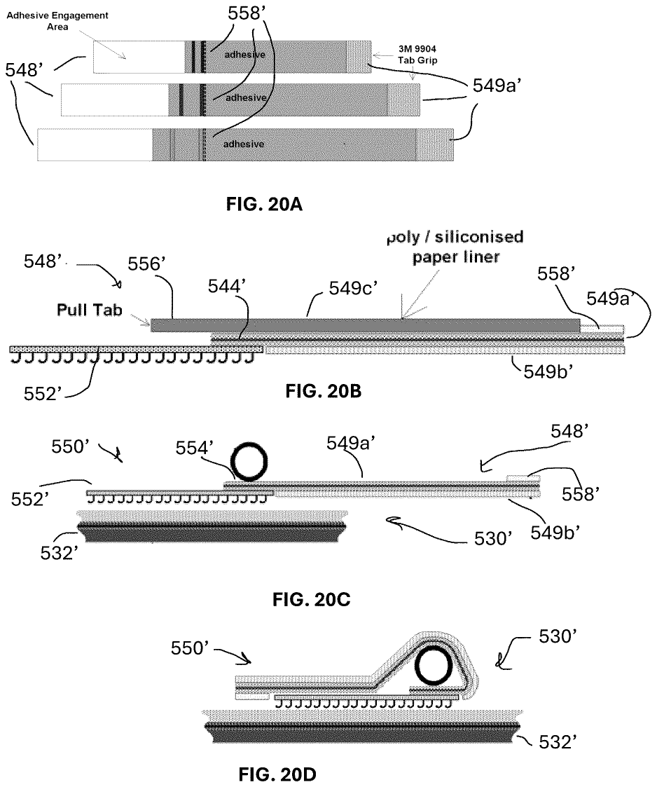

FIGS. 20A-D show an alternative strip type device 530′ which includes a base 532′ and an attachment element 550′. The base 532′ is identical to the base 532 of FIGS. 19A-B, and will not be described further. The attachment element 550′ comprises a fastener 552′ and an extension or finger 548′. The fastener 552′ is identical to the fastener 552 of FIGS. 19A-B. The extension 548′, however, is slightly varied from the extension 548 of the device 530. In this version, the extension 548′ is comprised of a polymer film 549a′ having an upper surface covered with an adhesive. The polymer film 549a′ has a lower liner 549b′ covering all of lower surface of the film, except for a forward end of the film. The lower surface of the forward end of the film extends over the fastener 552′ to define an overlap area 554′ which is adhered to the upper surface of the fastener at the first end of the fastener 552′. An upper liner 549c′ covers the adhesive surface of the film 549a′ starting from a point forward of the end remote from the overlap area, and extends beyond the overlap area to define a pull tab 556′. The back end of the film 549′ is covered with a tab grip 558′. Use of the device 530′ is substantially identical to use of the device 530, as discussed above. However, as can be appreciated, prior to placing the tubing T on the overlap area, the upper liner 549c′ is removed.

To help with positioning of the tubing T on the extensions, the extensions 548′ are provided with indicia 558′ in the form of two spaced apart lines that are positioned in the overlap area 554′, as seen in FIG. 20A. As is known, medical tubing is typically color coded, with each color designated a different sized tubing. The indicia 558′ on the extensions are preferably provided with the same color coding as the tubing. Thus, for example, an attachment element 550′ having green indicia will be intended for use with a green coded IV tube (which is an 18 gauge tube). Additionally, the two lines of the indicia are spaced apart a distance corresponding to the outer diameter of the tubing the extension 548′ is intended to be used with. Thus, the tubing can be placed between the two lines for proper placement of the tubing on the device 530′. As seen in FIG. 20A, the extensions 548′ have different lengths, based on the size (gauge) of tubing with which the extension is designed to be used. Thus, the extension sized to be used with a large bore tubing is longer than the extension sized to be used with the small bore tubing.

Because the attachment elements are color coded, a medical practitioner can quickly select the proper attachment element to be used. Further, because the attachment element 550, 550′ is separate from the base 532, 532′, a hospital or other medical facility can stock the bases 532, 532′ separately from the attachment elements 550, 550′. This enables the hospital to separately gauge the number of bases 532, 532′ and attachment elements 550, 550′ it will need, reducing the hospitals cost in stocking product, as it will not need a base for each attachment element.

Although described as a stand-alone tube-securing device, it will be appreciated that the attachment elements 550, 550′ could be used with the device of, for example, FIG. 2, to secure tubing to a patient's face. In this instance, the device of FIG. 2 would be formed without the fingers 48 as they would be superfluous. In this instance, the attachment elements 550 would be mounted to the body 32 of the device 30 by means of the fastener 552, 552′.

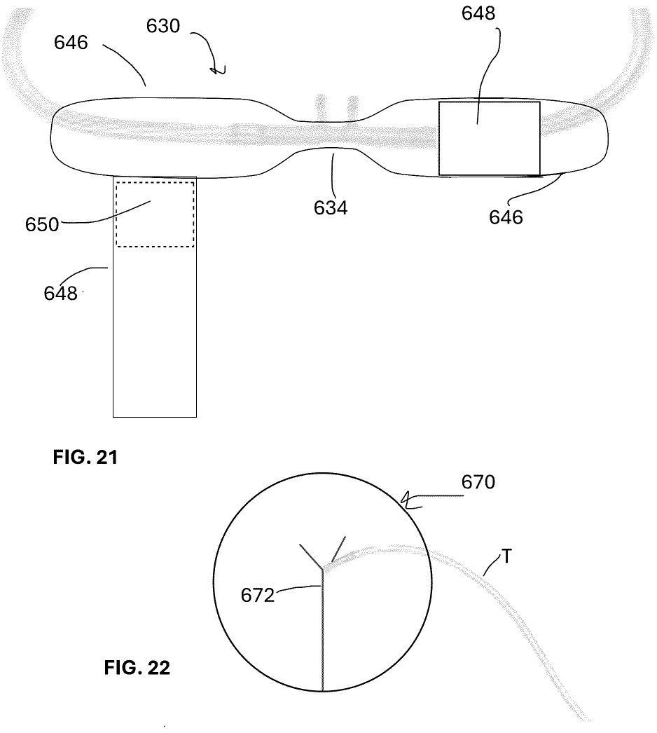

FIG. 21 shows a thin tube-securing device 630 designed to be used with a nasal cannula. The tube-securing device 630 is made from the adhesive material 20. It comprises a bridge section or body 634 which is sized to fit between the patient's nose and lip. Elongate wing or cheek portions 646 extend from the opposite ends of the bridge section 634. The bridge section 634 is preferably at least as long as the patient's nose is wide, so that the wings 646 will not engage either the patient's nose or mouth. Each wing 646 has a finger 648 extending from a long edge thereof. The finger 648 on the right cheek portion is shown holding tubing in place. A section of hook material 650 is adhered to the top surface of the fingers 648 proximate a base of the fingers, where the fingers join the wings. The use of the device 630 is generally similar to the use of the device 30, as described above. Initially, the liner of the material is removed from the device 630, and the device is positioned on a patient's face below the patient's nose. The finger 648 is folded over, so that the hooks of the hook material 650 will engage the plush surface of the wing/cheek portion, to hold the finger 648 in the folded over position. At that point, tubing T is placed across the folded over finger in a direction generally transverse to the axis of the finger. In doing so, the tubing will be placed against the adhesive of the finger. The finger can then be folded over the tubing, to surround the tubing, as shown, for example, in FIG. 5. If desired, the device 630 could be provided with the finger in the folded over position. This would eliminate the need for the hook material 650.

FIG. 22 shows an adhesive device 670 formed form the adhesive material 20. The device 670 is shown to be round, but could be any desired shape. The device includes a Y-shaped slit 672 which passes through at least the barrier material 22. The slit 672 can have an end (such as the bottom of the leg of the Y that extends to the edge of the device. In use, the tubing T will pass through the slit, at the top of the leg and the base of the “V” portion of the Y. The device 670 can help stabilize a Gastrostomy or Jejunostomy tube.

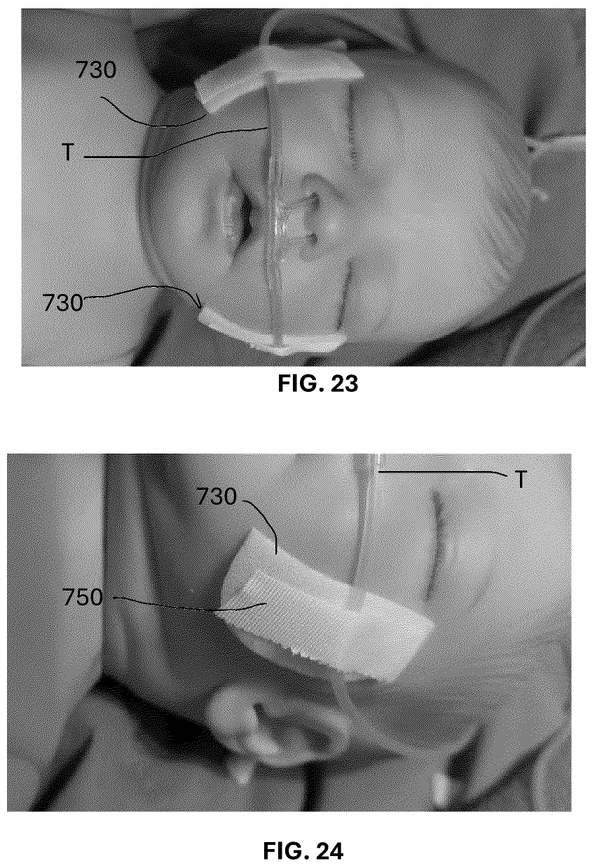

FIGS. 23 and 24 show a much simplified tube-securing device 730 adhered to the cheeks of a baby-doll. The tube-securing device 730 of FIGS. 23 and 24 comprise a piece of the adhesive material 20, which is adhered to each cheek. As seen, the devices 730 can be generally semi-circular. After the tubing T is positioned on the tube-securing device 730, a piece of surgical tape 750 is applied to the tube-securing device 730 over the tubing T. The medical tape 750 adheres well to the adhesive material 20, and thus, a simple piece of the adhesive material 20 when used in conjunction with medical tape, forms a suitable tube-securing device.

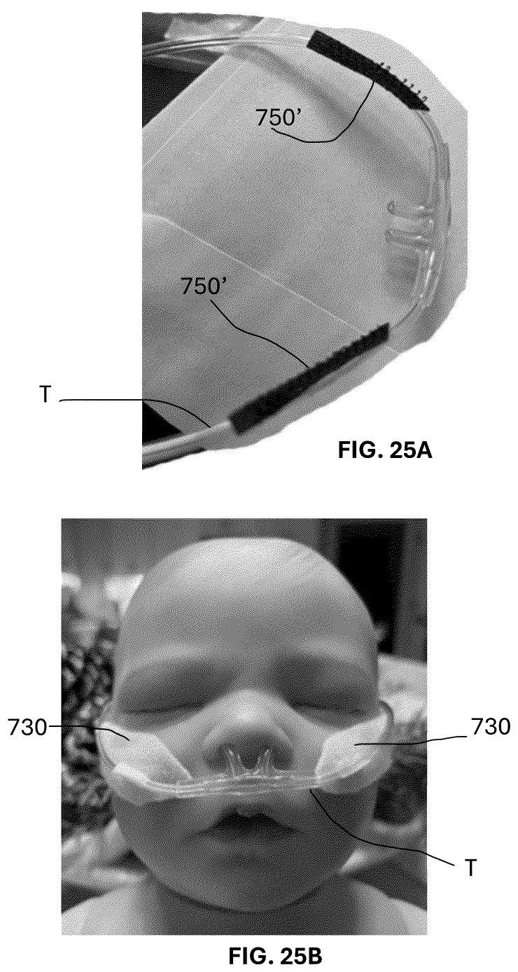

FIGS. 25A-B show a slight variation which avoids the need for the tape 750. In this variation, a hook material 750′ is adhered directly to the tubing, so that tubing T can be held to the device 730 by the hooks of the hook material 750′. In a variation, surgical tape (micropore), cut to size and taped to tubing can engage the plush or pile surface of the device 730 to hold the tubing T to the device.

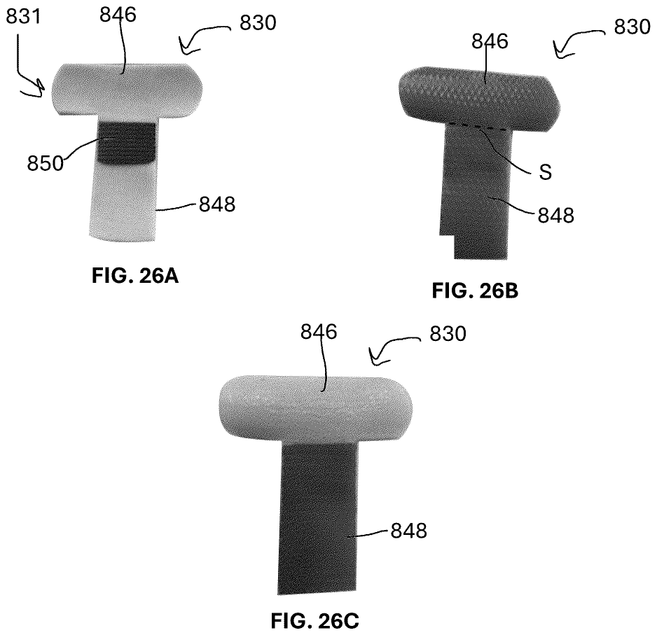

A seventh tube-securing device 830 is shown in FIGS. 26A-C. The tube-securing device 830 includes a body 831 comprising a cheek portion 846 which is generally rectangular in shape, but can be any desired shape. A single finger 848 extends from the approximate center of the cheek portion 846 and includes an attachment element 850 proximate the base of the finger 848. As seen in FIG. 26B, the release liner is slit, as at S, at the base of the finger 848. The slit S extends only through the release liner, so that when the release liner is removed from the cheek portion 846, the release liner will remain on the finger 848, as shown in FIG. 26C.

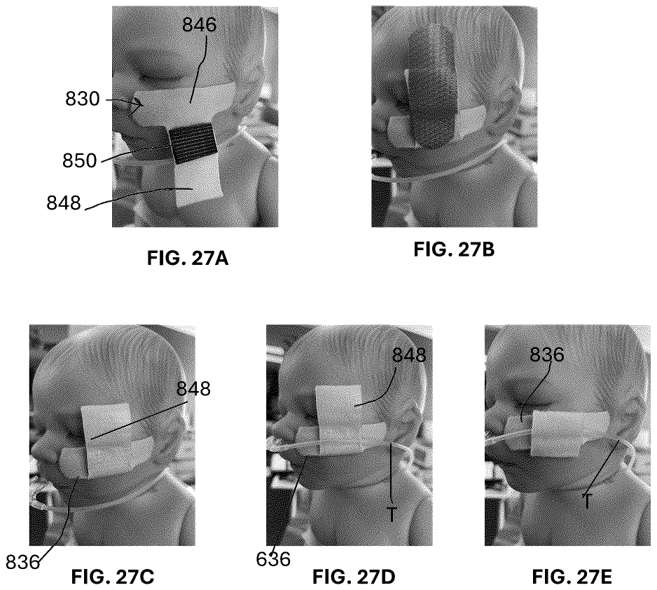

Use of the tube-securing device 830 is shown in FIGS. 27A-E. Initially, the backing is removed from the cheek portion 846 and the cheek portion is adhered to the patient's cheek, as seen in FIG. 27A. The finger 848 is then folded up, as seen in FIG. 27B, and the attachment element 850 is adhered to the upper surface of the cheek portion 846. The release liner is then removed from the finger 848, as seen in FIG. 27C and the tubing is positioned to extend along the length of the cheek portion, as seen in FIG. 27D. The finger 848 is then folded about the tubing or tube T to adhere to itself. Thus, the method of use is generally similar to the method of use of the tube-securing device 130 (of FIG. 7).

As can be appreciated, by making the above noted devices from the noted adhesive material 20 we have provided an adhesive device which can be used to securely position medical tubing, such as cannulas, NG tubes, IVs etc. on patients and which will reduce the potential, relative to currently available devices, of damaging the patient's skin. More importantly, the device is relatively easy to use.

As various changes could be made in the above constructions without departing from the scope of the invention, it is intended that all matter contained in the above description or shown in the accompanying drawings shall be interpreted as illustrative and not in a limiting sense. For example, the tube-securing device 130 (FIGS. 7-9) could be provided with slits to form a loop similar to the loop of the device 230 (FIG. 10). This would be in lieu of the hook material which forms the attachment element 150 for the device 130. In the embodiments of FIGS. 2 and 7, the fingers 48a,b and 148 are shown as extending from the body of the tube-securing device. The device could be formed so that the fingers in contained within the body or end at an edge of the body. Such a configuration, however, would require a substantially larger body, which would then cover more of the patient's skin. These examples are merely illustrative.

1. A tube-securing device for securing medical tubing in place on a patient; the tube-securing device comprising a body adapted to be adhered to skin of a patient and having at least one tube securing element; said tube securing element being adapted to retain medical tubing in place relative to the tube-securing device; said tube-securing device being formed from a material having an adhesive lower surface and a napped, plush or piled upper surface;
said at least one tube-securing element comprising one of:
a finger having a fixed end and a free end; said finger being pivotal relative to said body about said fixed end; wherein said finger is adapted to engage the medical tubing when in use;
a finger mounted to, and extending from, a fastener, said fastener being removably connectable to said body; or
a loop defined by a pair of generally parallel slits in the body of said tube securing device; said loop being sized such that the tubing can pass under the loop when the loop is raised out of a plane of the body.

2. The tube-securing device of claim 1 wherein the tube-securing device is made of a material comprising:
a top layer; the top layer comprising a base material, an upper pile extending from an upper surface of said base material, and a lower pile extending from a lower surface of said base material; said base material being flexible and stretchable; said upper pile defining said napped upper surface of said device;
a reinforcing layer, said reinforcing layer being secured to said top layer by a first binder which will bond to said reinforcing layer; said lower pile of said top layer extending into said binder; and
an adherent layer comprising a silicone gel, said silicone gel being operatively bound to said reinforcing layer by a second binder; said binder being selected to be a binder, which will bond to the silicone gel and to the reinforcing layer; said silicone gel defining said adhesive lower surface.

3. The tube-securing device of claim 1 wherein said finger extends from said body such that said fixed end is at an edge of said body, or
wherein said finger fixed end is spaced from said edge of said body, and said free end is spaced from or proximate said edge of said body.

4. The tube-securing-device of claim 3 further including an attachment element; said attachment element adapted to engage the napped surface of said body to maintain said finger in a position wherein said finger is folded about the fixed end of said finger to secure said finger against said body such that the adhesive lower surface of said finger faces away from said body and such that an outer portion of said finger is adapted to fold over in a direction opposite from the fold about said finger fixed end, whereby said finger is adapted to define a loop which, in use, surrounds the medical tubing.

5. The tube-securing device of claim 4 wherein said attachment element comprises one of a hook-type material positioned either between said fixed end and free end of said at least one finger or at the fixed end of said finger;
wherein said hook-type material is adapted to engage the napped upper surface of said fixed end or a strip defined by two opposed generally parallel slits in said body, said strip extending generally perpendicular to said at least one finger.

6. The tube-securing device of claim 5 wherein said strip is fixed at its opposite ends, whereby said strip defines a loop through which said finger can extend.

7. The tube-securing device of claim 1 wherein said device comprises a nose-surround and at least one cheek portion extending from a right or left side of said nose-surround.

8. The tube-securing device of claim 7 wherein said device comprises two cheek portions, there being a cheek portion extending from opposite sides of said nose-surround.

9. The tube-securing device of claim 1 including a plurality of said tube-securing elements or including only a single tube-securing element.

10. The tube-securing device of claim 1 wherein said device is light transmissive.

11. An adhesive device for securing medical equipment to the face of a patient, the adhesive device being formed from a material having an adhesive lower surface and a napped upper surface, said adhesive device comprising:
a nose-surround comprising a base sized to be positioned between an upper lip and nose of a patient and arms extending from opposite ends of said base; said base and arms defining an opening sized to fit over the nose of a patient; and
a cheek portion extending from each side of said nose surround.

12. The adhesive device of claim 11 wherein said adhesive device is adapted to have medical tubing secured thereto or to have a nasal Continuous Positive Airway Pressure (CPAP) mask secured thereto.

13. The adhesive device of claim 11 further comprising an upper structure ; said upper structure comprising a bridge member extending from an apex of said nose surround; said bridge member being sized to extend along a bridge of the nose of the patient.

14. The adhesive device of claim 13 wherein the upper structure further includes a cross-bar extending across a top of said bridge member.

15. A tube-securing device adapted to secure medical tubing to a patient; the tube securing device comprising:
a base; said base being formed from a pliable, flexible, conformable material having an adhesive lower surface and a napped upper surface; and
an attachment element removably securable to said base; said attachment element comprising:
an elongate fastener; said fastener having hook elements along a bottom surface thereof; said hook elements being adapted to removably engage the napped upper surface of said base to removably hold said attachment element to said base; and
a finger extending from a first end of said fastener; said finger comprising a film having an adhesive layer on an upper surface thereof; said finger having a length such that said finger can be folded over tubing positioned on said fastener and extend along a majority of the length of said fastener.

16. The tube-securing device of claim 15 wherein said attachment element comprising positioning indicia which indicate a position on said attachment element for placement of medical tubing.

17. The tube-securing device of claim 16 wherein said positioning indicia is color coded; the color of said indicia indicating a size of tubing for which the attachment element is designed to be used.

18. The tube-securing device of claim 16, wherein said positioning indicia comprises a pair of generally parallel spaced apart lines; said lines extending generally perpendicular to an axis of said attachment element.
